(19) United States
(12) Patent Application Publication
(10) Pub. No.: US 2026/0327182 A1
(43) Pub. Date: Oct. 22, 2026

(54) METHOD OF OPERATING A HYDROGENATION REACTOR

(71) Applicant: Covestro Deutschland AG, Leverkusen (DE)

(72) Inventors: Bernd Pennemann, Bergisch Gladbach (DE); Thomas Bludowsky, Ratingen (DE)

(21) Appl. No.: 19/115,838

(22) PCT Filed: Sep. 27, 2023
(86) PCT No.: PCT/EP2023/076768

§ 371 Date: Mar. 27, 2025

(30) Foreign Application Priority Data
Sep. 30, 2022 (EP) 22199186.2
Jun. 22, 2023 (EP) 23180816.3

(51) Int. Cl. C07C 209/36; B01J 19/00

(52) U.S. Cl. CPC C07C 209/36; B01J 19/0006; B01J 2219/00191

(57) ABSTRACT

The present invention relates to a method of operating a reactor for continuous hydrogenation of aromatic nitro compounds, specifically for closed-loop control thereof with regard to the completeness of conversion in the event of increases in throughput, for example in the startup phase after startup of the reactor up to attainment of a steady operating state. The method is more particularly characterized in that, over a period Δt₁₀ within which an increase in throughput is implemented, (a) the flow rate of aromatic nitro compound N(Ar(NO₂)ₘ) fed to the reactor and (2) the difference between the flow rate of hydrogen fed to the reactor and the flow rate of any hydrogen removed from the reactor, ΔN(H₂)=N(H₂)−N^{AUS}(H₂), is determined n times, where n is a natural number in the range from 10 to 3000, wherein the supply of aromatic nitro compound to the reactor is stopped when a theoretical value, calculated by the summation (I) elucidated hereinafter after each of the n determinations, for an (absolute) amount of non-hydrogenated aromatic nitro compound N^{FIK}(Ar(NO₂)ₘ) in the liquid phase (F) reaches or exceeds a maximum value N_{MAX}^{FIK}(Ar(NO₂)ₘ) fixed beforehand: (I), in which i is a serial index with which the n determinations are numbered, Nᵢ(Ar(NO₂)ₘ) is reported as the mass flow rate nᵢ(Ar(NO₂)ₘ) (for example in mol·h⁻¹), S represents a stoichiometric factor, with S=3·m, ΔN_{I}(H₂) is reported as the mass flow rate Δnᵢ(H₂) (for example in mol·h⁻¹), and Δtᵢ(Ar(NO₂)ₘ) and Δtᵢ(H₂) each represent the period between two successive determinations.

The present invention relates to a method of operating a reactor for continuous hydrogenation of aromatic nitro compounds, specifically for closed-loop control thereof with regard to the completeness of conversion in the event of increases in throughput, for example in the start-up phase after commencement of operation of the reactor until attainment of a steady state of operation.

It is a feature of continuously conducted chemical reactions that, in regular operation, a particular flow rate of reactants is fed continuously to a reactor (the reactor is “loaded” with a particular flow rate of reactants; is operated with a particular “load” of reactants), and a corresponding flow rate of products is withdrawn continuously from the reactor. Continuously operated chemical reactors are designed and optimized for operation at a particular flow rate of reactants; this flow rate is often referred to as “nameplate load”. During the operation of such a reactor, there are regularly various periods in which, for the purpose of increasing the flow rate of products obtained (production volume for short hereinafter), the flow rate of reactants which is supplied to the reactor has to be increased (i.e. an increase in “load”, or in other words an increase in throughput, is undertaken), for example in the start-up of a reactor after a shutdown period or if a reactor has been temporarily operated at lower than “nameplate load” (for instance owing to lack of demand for product) and the “load” is to be increased to “nameplate load”. The method of the invention is concerned with such periods of increasing throughput to increase the production volume. The method is more particularly characterized in that, during a period Δt₁₀ within which an increase in throughput is undertaken,
(1) the flow rate of aromatic nitro compound supplied to the reactor, N(Ar(NO₂)ₘ), and
(2) the difference between the flow rate of hydrogen supplied to the reactor and flow rate of any hydrogen removed from the reactor, ΔN(H₂)=N(H₂)−N^{OUT}(H₂), are determined n times where n is a natural number in the range from 10 to 3000, wherein the feed of aromatic nitro compound into the reactor is interrupted when a fictive value for an (absolute) amount of non-hydrogenated aromatic nitro compound N^{FIK}(Ar(NO₂)ₘ) in the liquid phase (L), calculated according to the summation (I) explained below after each of the n determinations, attains or exceeds a previously fixed maximum permissible value N_{MAX}^{FIK}(Ar(NO₂)ₘ):

$${n}_{i}^{FIK} ( {Ar ( {NO}_{2} )}_{m} ) = {\sum }_{i = 1}^{i = n} \{ {N}_{i} ( {Ar ( {NO}_{2} )}_{m} ) \cdot Δ {t}_{i} ( {Ar ( {NO}_{2} )}_{m} ) \} - S \cdot {\sum }_{i = 1}^{i = n} \{ Δ {N}_{i} ( {H}_{2} ) \cdot Δ {t}_{i} ( {H}_{2} ) \} \qquad ( I )$$

in which
i is a serial index by which the n determinations are serially numbered, Nᵢ(Ar(NO₂)ₘ) is reported as the molar flow rate \dot{n}ᵢ(Ar(NO₂)ₘ) (for example in mol·h⁻¹),
S is a stoichiometric factor, with S=3·m,
ΔNᵢ(H₂) is reported as the molar flow rate Δ\dot{n}ᵢ(H₂) (for example in mol·h⁻¹), and
Δtᵢ(Ar(NO₂)ₘ) and Δtᵢ(H₂) each represent the period between two successive determinations.

The hydrogenation of aromatic nitro compounds to the corresponding aromatic amines according to the equation

Ar(NO₂)ₘ+3 m H₂→Ar(NH₂)ₘ+2 m H₂O

is an important reaction in preparative organic chemistry, and is also of great significance in industrial production. Aniline (ANL) and tolylenediamine (TDA), which are probably the most important aromatic amines with regard to annual production volume, are both produced predominantly to completely by hydrogenation of the corresponding nitro compounds, mononitrobenzene (MNB; Ar═C₆H₅; m=1) and dinitrotoluene (DNT; Ar═CH₃C₆H₃; m=2). For this purpose, hydrogenation reactors have to be operated continuously with a high production capacity (and consequently a high throughput), which presents challenges in terms of process control technology. In this regard, both safety aspects and quality aspects have to be considered. In particular, an inadequate conversion of aromatic nitro compound can be problematic in both aspects.

WO 2006/089906 A1 describes a process for producing aromatic amines by hydrogenation of nitroaromatics or of aliphatic amino alcohols by hydrogenation of nitro alcohols in the presence of catalysts, wherein a fluid reaction mixture containing amines or amino alcohols is formed in a reactor, and the catalysts are separated therefrom. The process is more particularly characterized in that, after the catalyst has been separated off, a measurement of absorption of UV/VIS radiation by the reaction mixture is conducted to determine the concentration of nitro and nitroso compounds in the reaction mixture. Monitoring of conversion via UV/VIS spectroscopy is naturally associated with measurement complexity. Moreover, such devices require regular monitoring since the precision of such optical test methods can be reduced with time, for example as a result of soiling of the measurement devices used, which can soil the windows required for the measurement and the like.

EP 1 077 921 A1, published as WO 99/59956 A1, describes a process for producing an optionally substituted 4-aminodiphenylamine, comprising the conversion of optionally substituted aniline and optionally substituted nitrobenzene in the presence of water and a base, where the water content is controlled such that a molar ratio of water to the base used of not less than about 4:1 is assured at the start of the coupling reaction and not less than about 0.6:1 at the end of the coupling reaction, in order to prepare 4-nitrodiphenylamine and/or 4-nitrosodiphenylamine and/or salts thereof. The coupling reaction is followed by a hydrogenation reaction in which the coupling reaction product is hydrogenated in the presence of a hydrogenation catalyst and added water, such that a molar ratio of total water to base of at least about 4:1 is assured at the end of the hydrogenation. Aqueous and organic phases are obtained, and the optionally substituted 4-aminodiphenylamine is obtained from the organic phase. Only batchwise process regimes are described specifically.

EP 2 523 933 A2, published as WO 2011/086050 A2, describes a process for preparing aromatic amines in the liquid phase by catalytic hydrogenation of the corresponding nitroaromatics in at least two reaction spaces connected in series, where at least one reaction space is operated isothermally and at least the reaction space downstream thereof is operated adiabatically, and, in preferred embodiments, the adiabatic temperature jump is used for monitoring of the reaction, which is possible only with difficulty, if at all, in the event of changes in the production volume. It appears that the procedure described is not applicable to processes that are operated purely isothermally or provide only one reaction space. Moreover, this process requires the reaction mixture to have a certain reactivity. If this is not the case, for instance owing to complete absence of catalyst in the event of incorrect operation or a technical defect, there is no adiabatic temperature jump, which makes corresponding reaction monitoring impossible.

EP 2 812 308 A1, published as WO 2013/117622 A1, describes a continuous process for preparing at least one aromatic amine by hydrogenation of at least one nitroaromatic with hydrogen, wherein a liquid phase containing at least the aromatic amine and a gas phase containing at least hydrogen are present, in the presence of a catalyst suspended in the liquid phase, at a temperature of 50 to 250 °C and a pressure of 5 to 50 bar. Reactors as disclosed in WO 00/35852 A1 are described as being particularly suitable. In the process according to WO 2013/117622 A1, the pressure in the reactor is kept essentially constant by continuous adjustment of the amount of hydrogen supplied to the reactor. The total amount of hydrogen supplied to the reactor is monitored and the feed of the at least one nitroaromatic is interrupted if the hydrogen uptake in the reactor is not at least 50 mol %, preferably at least 70 mol %, more preferably at least 90 mol %, even more preferably at least 95 mol %, especially at least 98 mol %, of the amount of hydrogen required for stoichiometric conversion of the at least one nitroaromatic to the at least one aromatic amine. A disadvantage of this process is that it is unsuitable for nonsteady states of operation as in particular in the case of increases in throughput, for example in the course of start-up of the hydrogenation reactor from a state of operation shutdown.

This is because a hydrogenation reactor is typically put into operation with flow rates of nitro compound and hydrogen that are significantly below the corresponding target flow rates. These starting flow rates are then increased gradually (for example increased continuously) until the desired target flow rates are attained. Only then is the hydrogenation reactor in a state of operation which is steady in the sense that-apart from conceivable operating faults-continuously defined and constant (apart from unintended fluctuations) flow rates of aromatic nitro compound and hydrogen are fed in, and defined constant flow rates of aromatic amine and water are removed. In the intermediate state before the first feeding of aromatic nitro compound and hydrogen until attainment of the target flow rates (start-up period), however, the flow rates are subject to constant changes. Even in periods in which no change is intended (“plateau phases” in the case of a stepwise increase in flow rates), fluctuations that are large compared to fluctuations in the steady state of operation can occur. In this sense, the start-up period is a nonsteady state of operation of the hydrogenation reactor. Monitoring of the hydrogenation in this state is associated with particular challenges that the present invention has addressed. In particular, it is very important to give plant operators a criterion on the basis of which a decision can be made, optionally in an automated manner, as to when a start-up process is at variance from the target procedure to such a degree that the feed of aromatic nitro compound must be stopped in order to be able to meet the requirements on the quality of the product or even safety requirements. The aforementioned aspects are significant not just in the start-up of a hydrogenation reactor from an operation shutdown but also in the case of increases in throughput during regular operation. It is conceivable, for example, that a hydrogenation plant after start-up is operated for a certain period of time with a throughput that does not correspond to the maximum possible throughput for the plant in question (for instance because demand for the product of the hydrogenation is not high enough). If demand then rises at a later point in time and the throughput is correspondingly increased, similar problems will arise to those in the case of start-up from a shutdown.

There was thus a need for further improvements in the field of hydrogenation of nitroaromatics. In particular, it would be desirable to be able to operate hydrogenation reactors such that, even in nonsteady states as exist in the case of increases in throughput, for example during start-up, reliable monitoring of the reaction is assured, especially with regard to the presence of unconverted nitro compound in the liquid phase. In this way, the reaction regime was to be improved in terms of quality and especially safety, and if at all possible without having to implement any great measurement complexity.

Taking account of this need, the present invention provides a method, specifically a computer-implemented method in particular, of operating a reactor (R) for continuous hydrogenation of an aromatic nitro compound (Ar(NO₂)ₘ) in periods of increasing throughput,
wherein the hydrogenation is performed in the presence of a catalyst with hydrogen (H₂) to obtain an aromatic amine (Ar(NH₂)ₘ) as follows:

Ar(NO₂)ₘ+3 m H₂→Ar(NH₂)ₘ+2 m H₂O
in which Ar is an aryl group and m is 1 or 2.

In the method of the invention, a liquid phase (L) containing a suspension of the catalyst and a gas phase (G) containing hydrogen are present in the reactor during the hydrogenation. The reactor is supplied with the aromatic nitro compound with a flow rate N(Ar(NO₂)ₘ) and with the hydrogen with a flow rate N(H₂), and a product stream (PS) containing the aromatic amine and any non-hydrogenated aromatic nitro compound is withdrawn while retaining the catalyst. Hydrogen is optionally removed from the reactor by purging with a flow rate N^{OUT}(H₂). A constant pressure is established in the gas phase (G).

The process comprises (at least) one increase in throughput within a period Δt₁₀ beginning at a time to and ending at a time t₁. An increase in throughput in the context of the present invention is considered to mean an increase in the flow rates of reactants supplied to the reactor, i.e. of aromatic nitro compound and hydrogen. In the event of an increase in throughput within the scope of the invention, the flow rate N(Ar(NO₂)ₘ) is increased continuously or at intervals from a value Nₜ₌ₜ₀(Ar(NO₂)ₘ) to a value Nₜ₌ₜ₁(Ar(NO₂)ₘ)>Nₜ₌ₜ₀(Ar(NO₂)ₘ) and the flow rate N(H₂) from a value Nₜ₌ₜ₀(H₂) to a value Nₜ₌ₜ₁(H₂)>Nₜ₌ₜ₀(H₂) (i.e. t₁ has been attained when the flow rates of aromatic nitro compound and hydrogen have each reached the target value for the end of the increase in throughput, although it is not necessary for both flow rates to attain the respective value simultaneously). An increase in throughput within the scope of the invention means in particular an increase in N(Ar(NO₂)ₘ) by at least 0.5%, more preferably by at least 1%, of the starting value, such that, in particular,

Nₜ₌₊ₜ₁(Ar(NO₂)ₘ)≥1.005·Nₜ₌ₜ₀(Ar(NO₂)ₘ)

and more preferably

Nₜ₌ₜ₁(Ar(NO₂)ₘ)≥1.01·Nₜ₌ₜ₀(Ar(NO₂)ₘ).

The flow rate of hydrogen supplied to the reactor, N(H₂) is increased here such that—taking account of any hydrogen discharge (called purge)—at least the stoichiometric amount of hydrogen required for the hydrogenation is always available. The maximum value of the increase in throughput is limited not just by the maximum production capacity of the reactor used.

It is a feature of the method of the invention that, during the period Δt₁₀,
(1) the flow rate of aromatic nitro compound supplied to the reactor, N(Ar(NO₂)ₘ), and
(2) the difference between the flow rate of hydrogen supplied to the reactor and flow rate of any hydrogen removed from the reactor, ΔN(H₂)=N(H₂)−N^{OUT}(H₂),
are determined n times, in particular n times by means of a sensor unit, where n is a natural number in the range from 10 to 3000,
   wherein the feed of aromatic nitro compound into the reactor is interrupted when a fictive value for an (absolute) amount of non-hydrogenated aromatic nitro compound N^{FIK}(Ar(NO₂)ₘ) in the liquid phase (L), calculated according to the summation explained below after each of the n determinations, attains or exceeds a previously fixed maximum permissible value N_{MAX}^{FIK}(Ar(NO₂)ₘ). It is appropriate here to represent the summation using molar amounts (n), so as to give N^{FIK}(Ar(NO₂)ₘ) as the molar flow rate n^{FIK}(Ar(NO₂)ₘ), where the maximum permissible value should of course also be expressed in the same unit, i.e. as a molar amount n_{MAX}^{FIK}(Ar(NO₂)ₘ):

$${n}_{i}^{FIK} ( {Ar ( {NO}_{2} )}_{m} ) = {\sum }_{i = 1}^{i = n} \{ {N}_{i} ( {Ar ( {NO}_{2} )}_{m} ) \cdot Δ {t}_{i} ( {Ar ( {NO}_{2} )}_{m} ) \} - S \cdot {\sum }_{i = 1}^{i = n} \{ Δ {N}_{i} ( {H}_{2} ) \cdot Δ {t}_{i} ( {H}_{2} ) \} \qquad ( I )$$

in which
i is a serial index by which the n determinations are serially numbered,
Nᵢ(Ar(NO₂)ₘ) is reported as the molar flow rate nᵢ(Ar(NO₂)ₘ) (for example in mol·h⁻¹),
S is a stoichiometric factor, with S=3·m,
ΔNᵢ(H₂) is reported as the molar flow rate Δnᵢ(H₂) (for example in mol·h⁻¹), and
Δtᵢ(Ar(NO₂)ₘ) and Δtᵢ(H₂) each represent the period between two successive determinations.

The present invention further provides a computer system for closed-loop control of the conversion of aromatic nitro compound (Ar(NO₂)ₘ) in the method of the invention, a computer program product and a production plant for continuous hydrogenation of an aromatic nitro compound (Ar(NO₂)ₘ) with hydrogen (H₂) in the presence of a catalyst, comprising the computer system of the invention.

Completely surprisingly, it has been found that the finding of a fictive value for an amount of non-hydrogenated aromatic nitro compound as elucidated above offers a suitable criterion for deciding, during an increase in throughput (for example during the start-up time), when the feed of aromatic nitro compound into the hydrogenation reactor has to be stopped for reasons of quality and/or safety. A suitable permissible maximum value can easily be determined by the person skilled in the art under given boundary conditions (type of reactor, operating conditions thereof, especially with regard to temperature and pressure, and the size of the desired target flow rates for aromatic nitro compound and hydrogen). Substantial to complete independence from the boundary conditions mentioned is obtained when a criterion for stoppage of the feed of aromatic nitro compound is fixed not using the absolute value but the concentration of the maximum permissible value of non-hydrogenated aromatic nitro compounds based on the mass of the liquid phase m_{F}. This is the subject of a preferred embodiment and will be elucidated in detail further down.

The process of the invention relates to closed-loop control of the reactor with regard to completeness of conversion in periods of increasing throughput. Such an increase in throughput comprises a period Δt₁₀ within which, beginning at a time t₀, the flow rate of aromatic nitro compound fed to the reactor and the flow rate of hydrogen fed to the reactor are increased (=increase in throughput). The time at which the two flow rates have attained the respectively desired elevated values is designated t₁ (i.e.: Δt₁₀=t₁−t₀). It is not necessary for the flow rates of nitroaromatic and hydrogen to attain the respectively desire elevated values simultaneously. If one of the two streams (especially the hydrogen stream N(H₂)) attains the value desired for the end of the increase in throughput later than the other stream, this later time corresponds to time t₁.

According to the invention, the reactor is operated continuously. This means that the reactants (i.e. aromatic nitro compound and hydrogen) and any additives used, such as solvents, are fed continuously to the reactor during a production cycle, and the products formed (i.e. aromatic amine and water) and additives (solvents) are withdrawn continuously from the reactor. This is not necessarily applicable to the catalyst used. The catalyst may, for example, be retained in the reactor by a filter and be exchanged only after a production cycle (or multiple production cycles) has ended, namely when the catalytic activity has decreased too much. However, a catalyst exchange (or else merely the supply of fresh catalyst) during continuous operation is likewise encompassed by the process of the invention. In the terminology of the present invention, a production cycle means the period from the start of continuous production until the end thereof. The commencement of continuous production is considered to mean the time at which the reactor is supplied for the first time with hydrogen and aromatic nitro compound (t_{B}, a special case of t₀). The end of continuous production is fixed in that the feed of the aromatic nitro compound is stopped (t₂). (In general, after the feed of aromatic nitro compound is stopped, the hydrogen is allowed to run for a while longer before the reactor is shut down completely in order to assure complete reaction of any aromatic nitro compound still present in the reactor.)

Flow rates, symbolized by N in the terminology of the present invention, may in principle be reported as mass flow rates, volume flow rates or molar flow rates. In industrial practice, nitroaromatic streams N(Ar(NO₂)ₘ) are often reported as mass flow rates m (Ar(NO₂)ₘ), for example in kg·h⁻¹. Hydrogen streams N(H₂) (no matter whether hydrogen supplied or removed) are often reported as volume flow rates Q(H₂), for example in m³(STP)·h⁻¹ (m³(STP)=standard cubic meters; the volume converted to the standard state of a pressure of 101 325 Pa and a temperature of 273.15 K). Either can also be employed without difficulty in the context of the present invention. For the sake of simplicity, the above equation (I) is formulated for molar flow rates \dot{n}ᵢ, so as to give the fictive value for the amount of non-hydrogenated aromatic nitro compound as the molar flow rate nᵢ^{FIK}(Ar(NO₂)ₘ). In the embodiment already mentioned with employment of the maximum permissible (fictive) concentration of non-hydrogenated aromatic nitro compound, this concentration is then found as the specific partial molar amount q_{MAX}^{FIK} (Ar(NO₂)ₘ). It is a simple matter for the person skilled in the art to reformulate the equation (I) in industrial practice for other units of flow rate with corresponding adjustment of the stoichiometric factor S, and the preferentially used concentrations in parameters other than the specific partial molar amount. This does not constitute a departure from the scope of the present invention.

The appended drawings show:

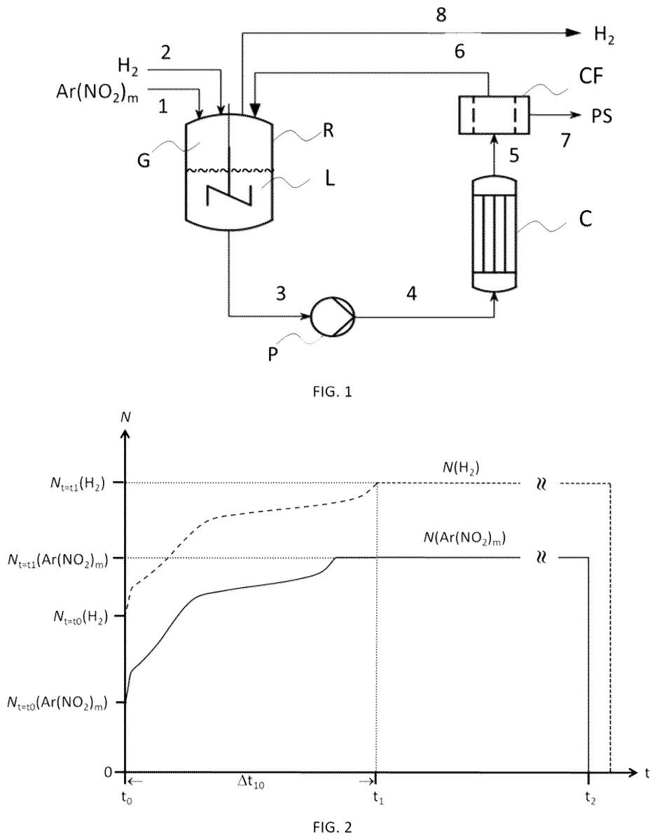

FIG. 1: a reactor including peripheral units that can be operated by means of the method of the invention;

FIG. 2 a schematic diagram of the flow rates N of reactants in a hydrogenation during a production cycle;

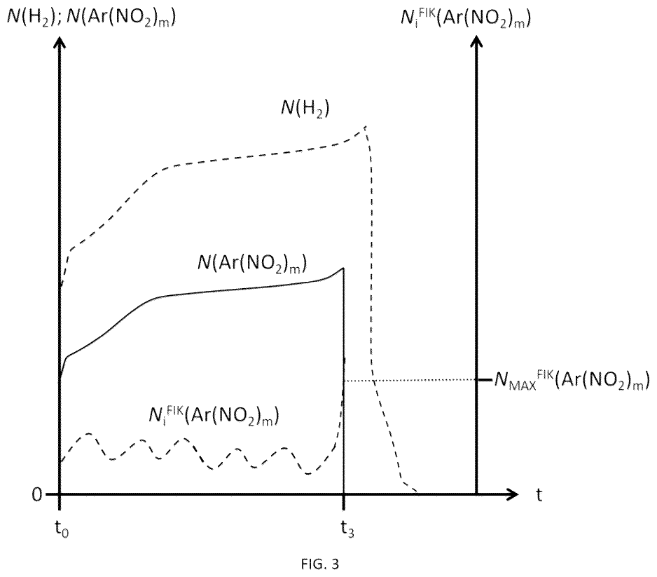

FIG. 3: a schematic diagram of the correlations between the fictive values ascertained for the amount of unreacted aromatic nitro compound and the stoppage of the feed of aromatic nitro compound;

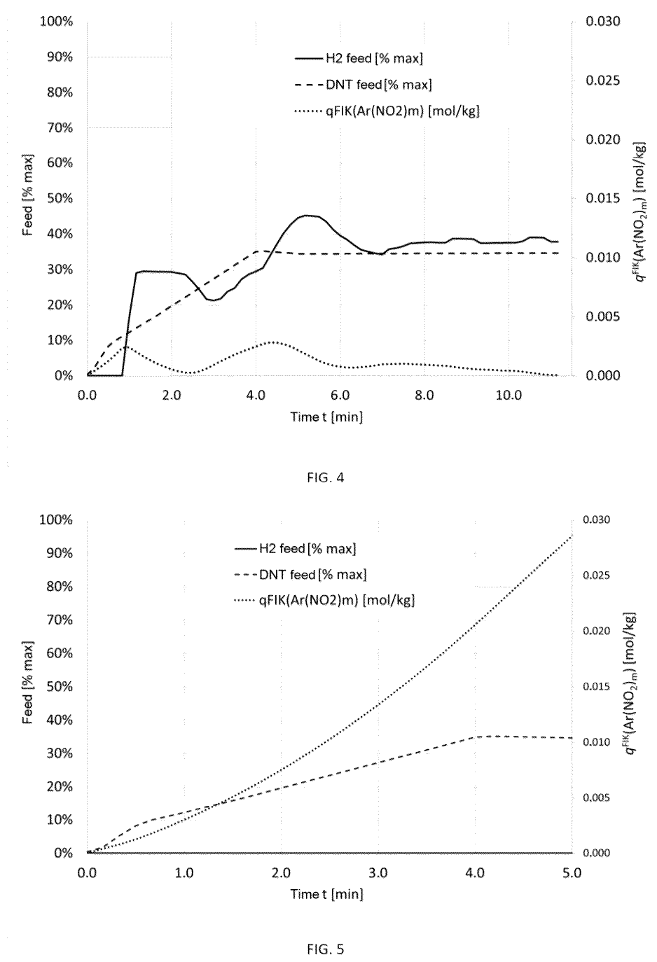

FIG. 4: the relative flow rates (in % of nameplate load) of hydrogen and dinitrotoluene (DNT) and the fictive concentration of non-hydrogenated aromatic nitro compound during a regular start-up procedure of a DNT hydrogenation plant; and

FIG. 5: the relative flow rates (in % of nameplate load) of hydrogen and dinitrotoluene (DNT) and the fictive concentration of non-hydrogenated aromatic nitro compound during a start-up procedure of a DNT hydrogenation plant in which the hydrogenation reactor is not supplied with any catalyst.

There will firstly follow a brief summary of various possible embodiments of the invention:

In a first embodiment of the method of the invention, which can be combined with all other embodiments, the specific partial molar amount q_{MAX}^{FIK}(Ar(NO₂)ₘ) of the maximum permissible value n_{MAX}^{FIK}(Ar(NO₂)ₘ) based on the mass m_{M_MAX}(L) of the liquid phase,

q_{MAX}^{FIK}(Ar(NO₂)ₘ)=n_{MAX}^{FIK}(Ar(NO₂)ₘ)/m_{M_MAX}(L),
is in the range from 0.006 mol/kg to 0.550 mol/kg, preferably 0.010 mol/kg to 0.200 mol/kg, more preferably 0.013 mol/kg to 0.029 mol/kg,
in which
m_{M_MAX}(L) is the mass of the liquid phase at the time in each determination i at which nᵢ^{FIK}(Ar(NO₂)ₘ) is for the first time equal to or greater than n_{MAX}^{FIK} (Ar(NO₂)ₘ) (and where each of the ranges specified for the specific partial molar amount q_{MAX}^{FIK}(Ar(NO₂)ₘ) is combinable with all other embodiments of the invention).

In a second embodiment of the method of the invention, which can be combined with all other embodiments, n is in the range from 80 to 3000 and the period of time Δt₁₀ is 5 min to 100 min. Preferably, n is in the range from 100 to 2500 and Δt₁₀ is 7.5 min to 80 min. More preferably, n is in the range from 500 to 2200 and Δt₁₀ is 15 min to 60 min. Most preferably, n is in the range from 1000 to 2000 and Δt₁₀ is 20 min to 40 min.

In a third embodiment of the method of the invention, which can be combined with all other embodiments, the nitroaromatic is mononitrobenzene (Ar═C₆H₅; m=1) or dinitrotoluene (Ar═CH₃C₆H₃; m=2).

In a fourth embodiment of the method of the invention, which can be combined with all other embodiments, the hydrogenation is conducted at a temperature in the range from 80 °C to 200 °C, preferably 110 °C to 180 °C, and at a pressure in the range from 5.0 bar to 120 bar, preferably 10 bar to 100 bar.

In a fifth embodiment of the method of the invention, which can be combined with all other embodiments, the catalyst comprises Pt, Pd, Rh, Ru, Ni, Co, Cu or mixtures thereof, preference being given to catalysts comprising Pt, Pd, Ni or Cu.

In a sixth embodiment of the method of the invention, which can be combined with all other embodiments, the reactor is a stirred tank reactor, a trickle film reactor or a bubble column reactor.

In a seventh embodiment of the method of the invention, which can be combined with all other embodiments, the suspension of the catalyst is a suspension in an organic solvent and/or in aromatic amine (Ar(NH₂)ₘ)) (from an earlier production cycle).

In an eighth embodiment of the method of the invention, which can be combined with all other embodiments, the increase in throughput comprises the commencing of the hydrogenation proceeding from a state in which the reactor (R) is not in operation.

The embodiments briefly outlined above and further possible configurations of the invention are elucidated in detail hereinafter. All the above-described embodiments and the further configurations of the invention described below are mutually and collectively combinable as desired unless the opposite is clearly apparent from the context to a person skilled in the art or is expressly stated.

Suitable aromatic nitro compounds are especially mononitrobenzene (Ar═C₆H₅; m=1) and dinitrotoluene (Ar═CH₃C₆H₃; m=2). The aromatic nitro compound is hydrogenated to the corresponding aromatic amine with hydrogen or mixtures of hydrogen and inert gases as hydrogenation reagent. The hydrogenation can be effected at temperatures and pressures that are customary in the specialist field. Preferred temperatures are in the range from 80 °C to 200 °C, especially 110 °C to 180 °C. Preferred pressures are in the range from 5.0 bar to 120 bar, especially 10 bar to 100 bar. (Here and hereinafter, all pressure figures should be considered to be absolute pressures.) The temperature is measured by means of devices known to the person skilled in the art, such as thermocouples or resistance thermometers, semiconductor thermometers or infrared thermometers. The pressure is preferably measured by mechanical manometers or electronic pressure sensors.

Catalysts used are all those used customarily for catalytic hydrogenations. Preference is given to using catalysts containing precious metals such as Pt, Pd, Rh, Ru or nonferrous metals such as Ni, Co or Cu or mixtures thereof. Particular preference is given to using catalysts containing Pt, Pd, Ni or Cu, in particular as a suspension in water. In the case of precious metal catalysts, these are applied to supports, for example activated carbon, SiO₂ or Al₂₀O₃; in the case of Ni catalysts, it is possible to use either supported Ni or a skeletal Ni catalyst. The concentration of the catalyst in the liquid phase present in the reactor is preferably 0.01% by mass to 20% by mass, preferably 0.50% by mass to 10% by mass, based on the total mass of the liquid phase.

If mixtures of hydrogen and inert gases are used, preferred inert gases are ammonia, noble gases and/or nitrogen. Hydrogen or the mixture of hydrogen and inert gases is applied so as to establish a constant pressure in the reactor, meaning that the amount of added hydrogen corresponds to the sum total of hydrogen consumed chemically and any hydrogen discharged as purge stream. In the case of use of a mixture of hydrogen and inert gases as hydrogenation reagent, the ratio of hydrogen and inert gas in the hydrogenation reagent supplied is increased gradually in order not to allow the reactor contents to become depleted of hydrogen.

In the method of the invention, hydrogen is preferably used in excess in relation to the hydrogenation of the nitro groups to amino groups required amount. In particular, the excess of hydrogen based on the molar amount required for the hydrogenation of the nitro groups to amino groups is at least 0.01%, preferably at least 0.10%, and not more than 10%. These values apply to steady states outside periods of increasing throughput. These values can generally also be used in periods of increasing throughput, but there may also be variances therefrom, especially in the case of increasing throughput for start-up of the reactor (commencement of operation).

It is optionally possible to use solvents that are inert under the reaction conditions, for example alcohols such as methanol, propanol, isopropanol, or ethers such as dioxane or tetrahydrofuran. In order to increase the economic viability of the method, a small solvent concentration is generally advantageous. This is typically 1.0% by mass to 50% by mass, preferably 20% by mass to 35% by mass, based in each case on the total mass of the liquid phase.

In addition, in the method of the invention, it is possible to discharge 0.10% to 10%, preferably 0.20% to 5.0%, of the flow rate of hydrogen used (purge hydrogen N^{OUT}(H₂)). This discharge can prevent the accumulation of inert compounds or gaseous by-products. These values apply to steady states outside periods of increasing throughput. These values can generally also be used in periods of increasing throughput, but there may also be variances therefrom, especially in the case of increasing throughput for start-up of the reactor (commencement of operation).

FIG. 1 shows, by way of example, a reactor that can be operated by means of the method of the invention. A stirred tank reactor is shown. Other suitable reactor types are in particular trickle film reactors and bubble column reactors.

To facilitate understanding of the present invention, operation of such a reactor in the steady state (i.e. in regular operation with constant throughput, especially with “nameplate load”) is described hereinafter:

The reactor (R) is supplied with the aromatic nitro compound (for example molten dinitrotoluene) with flow rate N_{TARGET}(Ar(NO₂)ₘ) via a nitroaromatics conduit (1) and with hydrogen at flow rate N_{TARGET}(H₂) via a hydrogen conduit (2). In the liquid phase (L) of the reactor (R) is a mixture of aromatic amine (i.e. tolylenediamine in the chosen example) and hydrogen, and also the catalyst, for example a nickel catalyst. The reactor (R) may be equipped with a device for feeding in fresh catalyst during operation (not shown in the figure). The aromatic nitro compound, i.e. dinitrotoluene in the example chosen, is hydrogenated with hydrogen at a temperature of, for example, 130 °C and a pressure of, for example, 20 bar.

A portion of the liquid phase (L) comprising the catalyst is conveyed in a circulation flow from the reactor (R) by means of a pump (P) via conduits 3, 4, 5 and 6. Conduit 4 opens into a cooler (C) in which the reaction mixture is cooled. Thence the cooled reaction mixture is fed via conduit 5 to a crossflow filter (CF), from which the product stream (PS) comprising the aromatic amine, i.e. tolylenediamine in the chosen example, is freed of catalyst and removed by the product conduit (7). The rest of the reaction mixture including catalyst is fed back to the reactor (R) via conduit 6, which completes the circuit.

N_{TARGET}(Ar(NO₂)ₘ) is adjusted so as to give an average residence time in the reactor (R) of, for example, 120 min. N_{TARGET}(H₂) is adjusted such that the pressure in the reactor (R) (measured in the gas space (G) above the liquid phase (L)) remains constant.

Hydrogen is removed continuously from the reactor (R) at a flow rate N_{TARGET}^{OUT}(H₂) via conduit 8 (purge). N^{OUT}(H₂) corresponds here, for example, to 1% of N_{TARGET}(H₂). FIG. 2 shows a schematic diagram of the flow rates N of reactants in a hydrogenation during a production cycle, showing the increase in throughput using the example of an increase in the flow rates of aromatic nitro compound and hydrogen from the start of the hydrogenation (t=t_{B}=t₀) until attainment of a steady state of operation (possibly a first steady state of operation) with constant throughput (from t=t₁). What is shown is an increase in throughput to start the hydrogenation proceeding from a state in which the reactor (R) is not in operation (start-up of the reactor), for which the method of the invention is particularly suitable.

The hydrogenation is to be started at time t_{B}=t₀, meaning that the reactor is supplied with hydrogen and nitroaromatic from this time. For preparation for the actual start-up (before time to; not shown in FIG. 2), the reactor is preferably charged with a suspension of the catalyst (i) in one of the abovementioned organic solvents that are inert under the conditions of the hydrogenation, (ii) in water and/or (iii) in aromatic amine (Ar(NH₂)ₘ)) (from an earlier production cycle) and heated up to the desired reaction temperature. The hydrogen feed is preferably likewise commenced before to, in such a way that the operating pressure desired for the steady state from t₁ is established in the gas phase (G). If a purge stream N^{OUT}(H₂) is provided, which is preferred, this is established at this early juncture.

From to, the reactor is then additionally supplied with the aromatic nitro compound. The starting values for the two flow rates, Nₜ₌ₜ₀(H₂) and Nₜ₌ₜ₀(Ar(NO₂)ₘ), are increased within a period Δt₁₀ to the desired values Nₜ₌ₜ₁(H₂) and Nₜ₌ₜ₁(Ar(NO₂)ₘ). In the diagram, the flow rate of aromatic nitro compound reaches the value envisaged for the steady state of operation (possibly the first) at an earlier stage than the hydrogen flow rate. However, the time t₁ is considered to have been attained only when both streams have reached the envisaged value. The period of time Δt₁₀ is preferably 5 min to 100 min, more preferably 10 min to 80 min, even more preferably 15 min to 60 min and very exceptionally preferably 20 min to 40 min. At time t₁, the flow rates are those desired for the steady state of operation (possibly the first). Then aromatic nitro compound is produced continuously in the reactor until the end of the production cycle at time t₂. The figure is not to scale; in general, the timespan t₂-t₁ will be several times the timespan t₁-t₀(=Δt₁₀). The feed of aromatic nitro compound is stopped at t₂, for example because demand for aromatic amine has dropped or the catalyst is exhausted. This stoppage need not be abrupt, as shown in the figure. A slow reduction is of course likewise possible. The timespan from t₀ to t₂ is referred to in the context of the present invention as production cycle.

It is possible that a production cycle comprises several different steady states of operation at constant throughput. For example, it is possible that the throughput of aromatic nitro compound and hydrogen at t₁ corresponds to a first steady state of operation, and that, after a certain period of time has elapsed, a second steady state of operation with further-elevated throughput is established. In such a case, the production cycle thus comprises two periods of time Δt₁₀: Δt₁₀ (1) and Δt₁₀ (2). In the case of such an existence of multiple periods of increasing throughput, the method of the invention is employed in at least one and preferably all these periods.

In the inventive procedure during at least one period Δt₁₀,
(1) the flow rate of aromatic nitro compound supplied to the reactor, N(Ar(NO₂)ₘ), and
(2) the difference between the flow rate of hydrogen supplied to the reactor and flow rate of any hydrogen removed from the reactor, ΔN(H₂)=N(H₂)−N^{OUT}(H₂),
are determined n times where n is a natural number in the range from 10 to 3000. The determination is especially effected by means of a sensor unit, for example by means of a conventional flowmeter.

The most suitable value of n depends on the duration of the period Δt₁₀. Preferably, n is in the range from
80 to 3000 when Δt₁₀ is 5 min to 100 min,
100 to 2500 when Δt₁₀ is 7.5 min to 80 min,
500 to 2200 when Δt₁₀ is 15 min to 60 min and
1000 to 2000 when Δt₁₀ is 20 min to 40 min.

The flow rate of aromatic nitro compound N(Ar(NO₂)ₘ) fed to the reactor at a particular time, since it is set to a particular value, is fundamentally known and can advantageously be monitored with a flowmeter for liquids and generally be read off directly on the process control system. The hydrogen is fed into the reactor under pressure control, meaning that any drop in pressure as a result of hydrogen consumption through progressive hydrogenation is counteracted by supply of a corresponding flow rate of hydrogen. This flow rate of hydrogen supplied, N(H₂), is determined with a flowmeter for gases. In principle, it is thus not absolutely necessary to remove hydrogen during a production cycle. As already mentioned, however, it has been found to be useful for removal of inerts and gaseous secondary components to remove some hydrogen from the reactor as a purge stream. The amount of hydrogen N^{OUT}(H₂)) discharged with the purge stream is determined by means of a flowmeter for gases or adjusted to a defined value by means of a perforated orifice plate or valve setting.

According to the invention, after each of the n determinations, a fictive value for the amount of non-hydrogenated aromatic nitro compound (=N^{FIK}(Ar(NO₂)ₘ)) is calculated. The resultant individual values Nᵢ^{FIK}(Ar(NO₂)ₘ) are added up, and as soon as the sum total reaches or exceeds a maximum permissible value fixed beforehand for the amount of non-hydrogenated aromatic nitro compound (=N_{MAX}^{FIK}(Ar(NO₂)ₘ)), the feed of aromatic nitro compound is stopped. This is shown schematically in FIG. 3:

The hydrogenation is commenced at to (as shown in FIG. 2). The time of increasing throughput, i.e. the start-up time in the example chosen, at first proceeds without difficulty. Although small fictive amounts of non-hydrogenated aromatic nitro compound are determined, these are at first still well below the maximum value fixed beforehand. However, this maximum value is attained at time t₃, and the supply of aromatic nitro compound is stopped. The time of stoppage is designated here as t₃ and not as t₂ by way of distinction from planned shutdown at the end of a production cycle.

As already mentioned, it is advantageous to express the maximum value permissible for non-hydrogenated aromatic nitro compound as a concentration, since a maximum value expressed in such a way is essentially independent of plant-specific parameters such as reactor size and the like. A useful reference parameter has been found to be the mass of the liquid phase (L). For this purpose, the fill level (which can in turn be established by means of a weir mounted at a suitable height or a closed-loop level control system) and the known reactor dimensions can be used to calculate the volume of the liquid phase, and from that in turn the mass thereof.

In this case, the maximum permissible value of non-hydrogenated aromatic nitro compound is thus expressed as the specific partial molar amount q_{MAX}^{FIK} (Ar(NO₂)ₘ). In particular, it has been found to be useful that

q_{MAX}^{FIK}(Ar(NO₂)ₘ)=n_{MAX}^{FIK}(Ar(NO₂)ₘ)/m_{M_MAX}(L) (II)
is in the range from 0.006 mol/kg to 0.550 mol/kg, preferably 0.010 mol/kg to 0.200 mol/kg, more preferably 0.013 mol/kg to 0.029 mol/kg, where, in equation (II),
m_{M_MAX}(L) is the mass of the liquid phase at the time in each determination i at which nᵢ^{FIK}(Ar(NO₂)ₘ) is for the first time equal to or greater than n_{MAX}^{FIK}(Ar(NO₂)ₘ) (and where each of the ranges specified for the specific partial molar amount q_{MAX}^{FIK}(Ar(NO₂)ₘ) is combinable with all other embodiments of the invention).

It is of course the case that the numerical values given for the specific partial molar amount q_{MAX}^{FIK}(Ar(NO₂)ₘ) should not be considered to be limiting in the sense that employment of different concentration parameters (for example proportions by mass) in industrial practice would result in departure from the scope of the invention for that reason alone. The numerical values given should instead be considered to be crucial in that concentrations, whatever unit they are given in in industrial practice, are covered by the numerical ranges given when they do so having been converted to specific partial molar amounts.

The present invention further provides a computer system for closed-loop control of the conversion of aromatic nitro compound (Ar(NO₂)ₘ) in the above-described method of the invention, where the computer system comprises the following:
an interface unit configured to import the results of the n-fold determination of N(Ar(NO₂)ₘ) and ΔN(H₂) over the period Δt₁₀, especially from the sensor unit described further up;
and
a processor configured to use the imported values for N(Ar(NO₂)ₘ) and ΔN(H₂) to calculate the molar amount nᵢ^{FIK}(Ar(NO₂)ₘ) and to compare it with the maximum permissible value n_{MAX}^{FIK}(Ar(NO₂)ₘ) that has been recorded in and can be called up from a database in communicative connection with the processor, and, on attainment or exceedance of the maximum permissible value n_{MAX}^{FIK}(Ar(NO₂)ₘ), to stop the feed of aromatic nitro compound into the reactor.

The computer system of the invention is preferably integrated into the process control system of the production plant.

The stoppage of the feed of aromatic nitro compound into the reactor on attainment or exceedance of the maximum permissible value n_{MAX}^{FIK}(Ar(NO₂)ₘ) is effected in particular in such a way that a stoppage signal is sent and received by a receiver unit (for example a valve controller), and then the feed of aromatic nitro compound is stopped (for example by closing an inflow valve).

The present invention further provides a computer program product comprising commands which, when the computer program product is executed by means of the above-described computer system of the invention, cause the computer system to execute the above-described method of the invention. In particular, this is a computer program product which, when loaded into the computer system of the invention, is executed by the processor of the computer system and, in the method of the invention, periodically or continuously calls up the results of the n-fold determination of N(Ar(NO₂)ₘ) and ΔN(H₂) and compares them with the maximum permissible value n_{MAX}^{FIK}(Ar(NO₂)ₘ) and, on attainment or exceedance of the maximum permissible value n_{MAX}^{FIK}(Ar(NO₂)ₘ), stops the feed of aromatic nitro compound into the reactor, especially as described above by sending a stoppage signal which is received by a receiver unit (for example a valve controller), and then the feed of aromatic nitro compound is stopped (for example by closing an inflow valve).

Finally, the present invention provides a production plant for continuous hydrogenation of an aromatic nitro compound (Ar(NO₂)ₘ) with hydrogen (H₂) in the presence of a catalyst to give an aromatic amine (Ar(NH₂)ₘ)) as follows:

Ar(NO₂)ₘ+3 m H₂→Ar(NH₂)ₘ+2 m H₂O
in which Ar is an aryl group and m is 1 or 2,
comprising:
(I) a reactor (R) for performance of the hydrogenation;
(II) an apparatus for provision of the aromatic nitro compound and introduction thereof into the reactor;
(III) an apparatus for provision of the hydrogen and introduction thereof into the reactor;
(IV) an apparatus for discharge of a product stream (PS) containing the aromatic amine and any non-hydrogenated aromatic nitro compound;
(V) an apparatus for retention of the catalyst on discharge of the product stream (PS); and
(VI) the computer system of the invention.

With regard to the apparatuses mentioned in (I) to (V), reference may be made to the above description of the method of the invention and the literature cited therein.

The invention is elucidated in detail hereinafter with the aid of examples.

### EXAMPLES

### Example 1

A plant for the production of tolylenediamine (TDA) from dinitrotoluene (DNT), comprising a stirred tank and an apparatus for separation and recycling of the catalyst from the product stream, was charged with the mixture of TDA and water formed in the hydrogenation (obtained from an earlier production cycle). The plant was brought to the required temperature, and a catalyst was added in accordance with an average concentration of about 1% by mass, based on the total mass of the reaction mixture. Subsequently, hydrogen was added to the plant by means of a pressure-retaining device until an absolute pressure of 25 bar was established in the gas phase. At time t=0, an amount of DNT increasing over time was added (see FIG. 4) until, after about 4.0 min, a value of about 35% of the maximum possible DNT stream for this plant is attained. After initial fluctuations, the hydrogen stream also reaches an essentially stable value after about 7.5 min (=t₁), which corresponds to about 37% of the maximum possible value for this plant. The calculated concentration q^{FIK}(Ar(NO₂)ₘ) of unreacted dinitrotoluene is well below 0.005 mol/kg at all times.

### Example 2

FIG. 5 shows the curve progression which is obtained when, for example, no catalyst is added to the reactor as a result of incorrect operation by operating personnel. The calculated concentration of unreacted dinitrotoluene increases here with time and already exceeds a value of 0.020 mol/kg after 4.0 min.

Example 1 shows that, in the present case, pure monitoring of the ratio can become active only after around 7.5 minutes owing to fluctuations in the pressure-retaining closed-loop control circuit. By contrast, example 2 shows that, by the present process, with choice of a maximum permissible concentration q_{MAX}^{FIK} (Ar(NO₂)ₘ) of 0.020 mol/kg, the impending hazardous enrichment of unreacted nitroaromatics is already recognized in virtually half the time.

1. A method of operating a reactor (R) for continuous hydrogenation of an aromatic nitro compound (Ar(NO₂)ₘ) in periods of increasing throughput,
wherein the hydrogenation is performed in the presence of a catalyst with hydrogen (H₂) to obtain an aromatic amine (Ar(NH₂)ₘ)) as follows:
Ar(NO₂)ₘ+3 m H₂→Ar(NH₂)ₘ+2 m H₂O
in which Ar is an aryl group and m is 1 or 2,
wherein, during the hydrogenation, a liquid phase (L) containing a suspension of the catalyst and a gas phase (G) containing hydrogen are present in the reactor, and the reactor is supplied with the aromatic nitro compound with a flow rate N(Ar(NO₂)ₘ) and with the hydrogen with a flow rate N(H₂), and a product stream (PS) containing the aromatic amine and any non-hydrogenated aromatic nitro compound is withdrawn from the reactor with retention of the catalyst, with optional removal of hydrogen from the reactor with a flow rate N^{OUT}(H₂) and with establishment of a constant pressure in the gas phase (G),
the method comprising an increase in throughput within a period Δt₁₀, beginning at a time t₀ and ending at a time t₁, said increase in throughput comprising an increase, continuously or at intervals, in the flow rate N(Ar(NO₂)ₘ) from a value Nₜ₌ₜ₀(Ar(NO₂)ₘ) to a value Nₜ₌ₜ₁(Ar(NO₂)ₘ)>Nₜ₌ₜ₀(Ar(NO₂)ₘ) and in the flow rate N(H₂) from a value Nₜ₌ₜ₀(H₂) to a value Nₜ₌ₜ₁(H₂)>Nₜ₌ₜ₀(H₂),
wherein, during the period Δt₁₀,
(1) the flow rate of aromatic nitro compound supplied to the reactor, N(Ar(NO₂)ₘ), and
(2) the difference between the flow rate of hydrogen supplied to the reactor and flow rate of any hydrogen removed from the reactor, ΔN(H₂)=N(H₂)−N^{OUT}(H₂), are determined n times where n is a natural number in the range from 10 to 3000, and
wherein the feed of aromatic nitro compound into the reactor is interrupted when a fictive value for a molar amount nᵢ^{FIK}(Ar(NO₂)ₘ) of non-hydrogenated aromatic nitro compound in the liquid phase (L), calculated according to the following equation after each of the n determinations, attains or exceeds a previously fixed maximum permissible value n_{MAX}^{FIK}(Ar(NO₂)ₘ):
$${n}_{i}^{FIK} ( {Ar ( {NO}_{2} )}_{m} ) = {\sum }_{i = 1}^{i = n} \{ {N}_{i} ( {Ar ( {NO}_{2} )}_{m} ) \cdot Δ {t}_{i} ( {Ar ( {NO}_{2} )}_{m} ) \} - S \cdot {\sum }_{i = 1}^{i = n} \{ Δ {N}_{i} ( {H}_{2} ) \cdot Δ {t}_{i} ( {H}_{2} ) \}$$
in which
i is a serial index by which the n determinations are serially numbered,
Nᵢ(Ar(NO₂)ₘ) is reported as the molar flow rate \dot{n}ᵢ(Ar(NO₂)ₘ),
S is a stoichiometric factor, with S=3·m,
ΔNᵢ(H₂) is reported as the molar flow rate Δ\dot{n}ᵢ(H₂), and
Δtᵢ(Ar(NO₂)ₘ) and Δtᵢ(H₂) each represent the period between two successive determinations.

2. The method as claimed in claim 1, in which the specific partial molar amount q_{MAX}^{FIK}(Ar(NO₂)ₘ) of the maximum permissible value n_{MAX}^{FIK} (Ar(NO₂)ₘ) based on the mass m_{M_MAX}(L) of the liquid phase,
q_{MAX}^{FIK}(Ar(NO₂)ₘ)=n_{MAX}^{FIK}(Ar(NO₂)ₘ)/m_{M_MAX}(L),
is in the range from 0.006 mol/kg to 0.550 mol/kg,
in which
m_{M_MAX}(L) is the mass of the liquid phase at the time in each determination i at which nᵢ(Ar(NO₂)ₘ) is for the first time equal to or greater than n_{MAX}^{FIK}(Ar(NO₂)ₘ).

3. The method as claimed in claim 1, in which n is in the range from 80 to 3000 and the period Δt₁₀ is 5 min to 100 min.

4. The method as claimed in claim 1, in which n is in the range from 100 to 2500 and Δt₁₀ is 7.5 min to 80 min.

5. The method as claimed in claim 1, in which n is in the range from 500 to 2200 and Δt₁₀ is 15 min to 60 min.

6. The method as claimed in claim 1, in which n is in the range from 1000 to 2000 and Δt₁₀ is 20 min to 40 min.

7. The method as claimed in claim 1, in which the nitroaromatic is mononitrobenzene or dinitrotoluene.

8. The process as claimed in claim 1, in which the hydrogenation is conducted at a temperature in the range from 80 °C to 200 °C

9. The process as claimed in claim 1, in which the catalyst comprises Pt, Pd, Rh, Ru, Ni, Co, Cu or a mixture thereof.

10. The process as claimed in claim 1, in which the reactor is a stirred tank reactor, a trickle film reactor or a bubble column reactor.

11. The process as claimed in claim 1, in which the suspension of the catalyst is a suspension in an organic solvent and/or in aromatic amine.

12. The process as claimed in claim 1, in which the increase in throughput comprises the commencing of the hydrogenation proceeding from a state in which the reactor (R) is not in operation.

13. A computer system configured for closed-loop control of the process as claimed in claim 1, comprising:
an interface unit configured to import in the results of the n-fold determination of N(Ar(NO₂)ₘ) and ΔN(H₂) over the period Δt₁₀;
and
a processor configured to use the imported values for N(Ar(NO₂)ₘ) and ΔN(H₂) to calculate the molar amount nᵢ^{FIK}(Ar(NO₂)ₘ) and to compare it with the maximum permissible value n_{MAX}^{FIK}(Ar(NO₂)ₘ) that has been recorded in and can be called up from a database in communicative connection with the processor, and, on attainment or exceedance of the maximum permissible value n_{MAX}^{FIK}(Ar(NO₂)ₘ), to stop the feed of aromatic nitro compound into the reactor.

14. A computer program product comprising commands which, on execution of the computer program product by a computer system, cause the computer system to execute the process as claimed in claim 1.

15. A production plant for continuous hydrogenation of an aromatic nitro compound
comprising:
a reactor (R) for configured for continuous hydrogenation of an aromatic nitro compound (Ar(NO₂)ₘ) with hydrogen (H₂) in the presence of a catalyst to give an aromatic amine (Ar(NH₂)ₘ)) as follows:
Ar(NO₂)ₘ+3 m H₂→Ar(NH₂)ₘ+2 m H₂O
in which Ar is an aryl group and m is 1 or 2;
an apparatus configured to provide the aromatic nitro compound and to introduce the aromatic nitro compound into the reactor;
an apparatus configured to provide the hydrogen and to introduce the hydrogen into the reactor;
an apparatus configured to discharge a product stream (PS) containing the aromatic amine and any non-hydrogenated aromatic nitro compound;
an apparatus configured to retain the catalyst on discharge of the product stream (PS);
and
the computer system as claimed in claim 13.
